(19) United States
(12) Patent Application Publication
(10) Pub. No.: US 2008/0255166 A1
(43) Pub. Date: Oct. 16, 2008

(54) Dithiocarbamate derivatives and their use as antibacterial agents

(75) Inventors: Ute Mollmann, Jena (DE); Vadim Makarov, Moscow (RU)

Correspondence Address: NIXON & VANDERHYE, PC, 901 NORTH GLEBE ROAD, 11TH FLOOR, ARLINGTON, VA, 22203, US

(73) Assignee: NEED PHARMACEUTICALS S.r.I., Milano (IT)

(21) Appl. No.: 12/155,604

(22) Filed: Jun. 6, 2008

Related U.S. Application Data
(62) Division of application No. 10/495,651, filed on Jul. 21, 2004, now abandoned.

(30) Foreign Application Priority Data
Nov. 17, 2001 (EP) 01 127 296 0

(51) Int. Cl. A61K 31/505; C07D 239/38; A61K 31/277; C07C 205/56; C07D 213/70; A61K 31/44

(52) U.S. Cl. 514/269; 544/298; 514/524; 560/22; 514/346; 546/291

(57) ABSTRACT

The invention relates to the use of dithiocarbamate derivatives for the therapeutic or prophylactic treatment of infectious diseases in mammals (humans and animals) caused by bacteria, especially diseases like tuberculosis (TB) and lepra caused by mycobacteria and infectious diseases caused by staphylococci. The invention further relates to novel dithiocarbamate derivatives of the formula (I), wherein X is a bivalent residue selected from the group consisting of formulae having excellent antibacterial activities, and to pharmaceutical preparations containing the same.

The invention relates to the use of dithiocarbamate derivatives, including novel dithiocarbamate derivatives, as antibacterial agents in infectious diseases of mammals (humans and animals) caused by bacteria, especially diseases like tuberculosis (TB) and lepra caused by mycobacteria and infectious diseases caused by staphylococci. The invention further relates to novel dithiocarbamate derivatives as such and pharmaceutical preparations containing the same.

As known, there is a threadful worldwide increase in tuberculosis infections with mycobacteria which developed resistance against the available therapeutics (B. R. Bloom, J. L. Murray, tuberculosis: commentary on a reemergent killer. Science 257, 1992, 1055-1064). Extremely dangerous is the development of multidrug resistant (MDR) mycobacteria. These are mycobacteria, resistant at least against two of the most active tuberculosis drugs, isoniazid and rifampicin, but also against streptomycin, pyranzinamid and ethambutol. The proportion of MDR-TB in some countries is already more than 20%. In Germany resistance against a single tuberculosis drug raised since 1996 for 50%.

Together with the increased number of diseases generally, worldwide tuberculosis causes a number of 3,000,000 deaths annually. Another growing problem is the therapy of infections with multiresistant staphylococci (M. Kresken, Bundesgesundheitsblatt 38,1996, 170-178).

For the treatment of such diseases there is an urgent need for new drugs with new mechanisms of actions, especially to overcome drug resistance and to overcome the known dramatic side effects of the available drugs.

The present invention aims at the generation of new compounds with activity against mycobacteria as potential new tuberculosis drugs and with activity against other Gram-positive pathogens like staphylococci to overcome problems concerning resistance and drug intolerance.

According to the present invention, it was surprisingly discovered that dithiocarbamate derivatives exhibit strong antibacterial activity, especially against mycobacteria

The aim is thus solved by the use of compounds of the general formula I

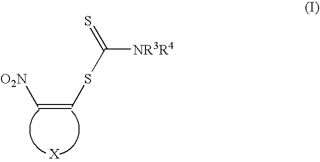

wherein X is a bivalent residue selected from the group consisting of

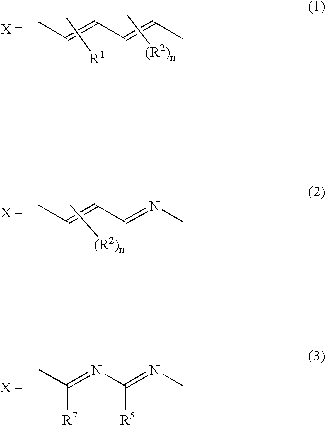

wherein

R¹ is CN, CONR³R⁴, CONHNR³R⁴;

R² is H, NO₂, CN, CONR³R⁴, CONHNR³R⁴, COOR⁵, CHO, halogen or a saturated or unsaturated, linear or branched aliphatic radical having 1-7 chain members, a saturated or unsaturated, linear or branched alkanol radical having 1-8 chain member, OR⁵, SR⁵, NR³R⁴, SO₂NR³R⁴, trifluoromethyl, phenyl;

n is 0-3;

R³ and R⁴ are, independently from each other, H, a saturated or unsaturated, linear or branched aliphatic radical having 1-7 chain members, cycloalkyl having 3-7 carbon atoms; a saturated or unsaturated, linear or branched alkanol radical having 1-8 chain member; benzyl, phenyl or naphthyl substituted by (R⁵)m wherein m is 0-7; OR⁵; or NR³R⁴ is morpholino, a saturated or unsaturated, mono or polyheterocyclic residue with heteroatoms N, S, O and substituted by (R⁵)m; or R³ and R⁴ together represent a bivalent radical —(CH₂)ₘ— wherein m is 2-7, or a bivalent radical —(CH₂CH₂)NR⁶(CH₂CH₂)—; or R³ and R⁴ together represent a bivalent radical

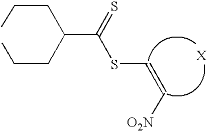

R⁵ is H, or a saturated or unsaturated, linear or branched aliphatic radical having 1-7 chain members, trifluoromethyl, benzyl or phenyl; or halogen, a saturated or unsaturated, halogenated or unhalogenated, linear or branched aliphatic radical having 1-7 chain members, a saturated or unsaturated, linear or branched alkanol radical having 1-8 chain members, benzyl, phenyl, stiryl or naphtyl, each unsubstituted or substituted by (R⁶)m; COOH, COOR⁵, CONR³N⁴;

R⁶ is H, halogen or a saturated or unsaturated, linear or branched aliphatic radical having 1-7 chain members, a saturated or unsaturated, halogetated or unhalogenated linear or branched alkanol radical having 1-8 chain member, trifluoromethyl, benzyl, phenyl, stiryl or naphtyl, each unsubstituted or substituted by (R⁶)m; COOH, COOR⁵, CONR³N⁴;

R⁷ is H, halogen or a saturated or unsaturated, linear or branched aliphatic radical having 1-7 chain members, OR⁵, SR⁵, NR³R⁴, trifluoromethyl, phenyl, stiryl, each unsubstituted or substituted by (R⁶)m;

for the manufacture of pharmaceutical preparations for the therapeutic or prophylactic treatment of bacterial infections, and pharmaceutical compositions containing the afore-mentioned compounds.

According to a particular embodiment, the above-mentioned compounds according to formula I are selected from the group consisting of

1,5-dinitro-3-cyano-6-dimethyldithiocarbamoyl-benzene, 3,5-dinitro-2-dimethyldithio-carbamoyl-pyridine, 3,5-dinitro-6-dimethyl-amino-2-dimethyl-dithiocarbamoyl-pyridine, 4-methoxy-6-diethyldithiocarbamoyl-5-nitro-pyrimidine, 4-methoxy-6-dipropyldithiocar-bamoyl-5-nitro-pyrimidine, 4-dimethyl-amino-6-diethyldithiocarbamoyl-5-nitropyrimidine, 4-dimethylamino-6-dipropyldithio-carbamoyl-5-nitro-pyrimidine, and 4-methyl-amino-6-diethyldithiocarbamoyl-5-nitro-pyrimidine.

Specifically, the invention relates to new compounds of the formula I

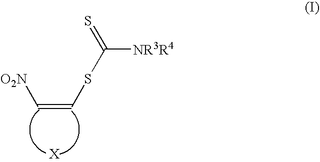

wherein X is a bivalent residue selected from the group consisting of

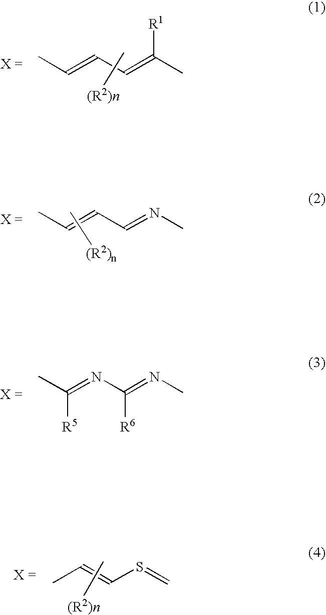

wherein

R¹ is CN, CONR³R⁴, CONHNR³R⁴;

R² is H, NO₂, CN, CONR³R⁴, CONHNR³R⁴, COOR⁵, CHO, halogen or a saturated or unsaturated, linear or branched aliphatic radical having 1-7 chain members optionally substituted by hydroxy; OR⁵, SR⁵, NR³R⁴, SO₂NR³R⁴, trifluoromethyl, phenyl;

n is 0-3;

R³ and R⁴ for X=1 are, independently from each other, H, an unsaturated, linear or branched aliphatic radical having 1-7 chain members, cycloalkyl having 3-7 carbon atoms; a saturated or unsaturated, linear or branched alkyl radical having 1-8 chain member substituted by hydroxy; benzyl, phenyl or naphthyl each unsubstituted or substituted by (R⁵)m wherein m is 1-5; OR⁵; or NR³R⁴ is a saturated or unsaturated, mono or polyheterocyclic ring with heteroatoms N, S, O which is unsubstituted or substituted by (R⁵)m;

R³ and R⁴ for X=2-4 are, independently from each other, H, a saturated or unsaturated, linear or branched aliphatic radical having 1-7 chain members optionally substituted by hydroxy; cycloalkyl having 3-7 carbon atoms; benzyl, phenyl or naphthyl each unsubstituted or substituted by (R⁵)m wherein m is 1-5; OR⁵; or NR³R⁴ is a saturated or unsaturated, mono or polyheterocycles with heteroatoms N, S, O and substituted by (R⁵)m ;

or R³ and R⁴ for X=1-4 together represent the bivalent radical

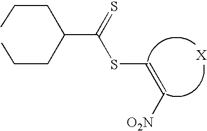

R⁵ is H, or halogen, a saturated or unsaturated, linear or branched aliphatic radical having 1-7 chain members optionally substituted by halogen or hydroxy; benzyl, phenyl, styryl or naphthyl each unsubstituted or substituted by (R⁶)m wherein m is 1-5; COOH, COOR⁵, CONR³N⁴;

R⁶ is H, halogen or a saturated or unsaturated, linear or branched aliphatic radical having 1-7 chain members optionally substituted by halogen or hydroxy; benzyl, phenyl, styryl or naphthyl each unsubstituted or substituted by (R⁶)m; COOH, COOR⁵, CONR³N⁴;

with the exclusion of
3,5-dinitro-2-dimethyldithio-carbamoyl-pyridine, 3,5-dinitro-6-dimethyl-amino-2-dimethyl-dithiocarbamoyl-pyridine, 4-methoxy-6-diethyldithiocarbamoyl-5-nitro-pyrimidine, 4-methoxy-6-dipropyldithiocar-bamoyl-5-nitro-pyrimidine, 4-dimethyl-amino-6-diethyldithiocarbamoyl-5-nitropyrimi-dine, 4-dimethylamino-6-dipropyldithio-carbamoyl-5-nitro-pyrimidine, and 4-methyl-amino-6-diethyldithiocarbamoyl-5-nitro-pyrimidine (Khim. Geterotsikl. Soedin., (Rus.) 1994, 10, p. 1420-1423, CA 122:314512), and 4-methylamino-6-diethyldithiocarbamoyl-5-nitro-pyrimidine (Acta Cryst. Section C, 2001, C57, p. 72-75),

and their use for the manufacture of pharmaceutical preparations for the therapeutic or prophylactic treatment of bacterial infections.

In a preferred embodiment, the invention concerns compounds of the formula (I) selected from the group consisting of
3,5-dinitro-2-R³R⁴-dithiocarbamoyl-benzonitriles,
3,5-dinitro-2-R³R⁴-dithiocarbamoyl-benzamides,
3,5-dinitro-2-R³R⁴-dithiocarbamoyl-pyridines, and
4-R⁷-2-R⁵-6-R³R⁴-dithiocarbamoyl-5-nitro-pyrimidines,
   wherein R³, R⁴, R⁵ and R⁷ have the above meanings,
   except 3,5-dinitro-2-dimethyl-dithiocarbamoyl-pyridine, 4-methoxy-6-diethyldithio-carbamoyl-5-nitro-pyrimidine, 4-methoxy-6-dipropyldithiocarbamoyl-5-nitro-pyrimidine, 4-dimethylamino-6-diethyldithiocarbamoyl-5-nitro-pyrimidine, 4-dimethylamino-6-dipropyl-dithiocarbamoyl-5-nitro-pyrimidine and 4-methylamino-6-diethyldithiocarbamoyl-5-nitro-pyrimidine.

The present invention is even more particularly concerned with at least one compound selected from the group consisting of
4-cyclopropylamino-6-diethyldithiocarbamoyl-5-nitropyrimidine,
4-methylhydroxyamino-6-diethyldithiocarbamoyl-5-nitropyrimidine,
4-ethoxy-2-methyl-6-diethyldithiocarbamoyl-5-nitropyrimidine,
4-methylamino-6-dimethyldithiocarbamoyl-5-nitropyrimidine,
4-dimethylamino-6-dimethyldithiocarbamoyl-5-nitropyrimidine,
4-ethoxy-2-methyl-6-tetramethylenedithiocarbamoyl-5-nitropyrimidine,
4-propoxy-6-dimethyldithiocarbamoyl-5-nitropyrimidine,
4-methylamino-6-tetramethylenedithiocarbamoyl-5-nitropyrimidine,
4-dimethylamino-6-tetramethylenedithiocarbamoyl-5-nitropyrimidine,
4-methoxy-6-dimethyldithiocarbamoyl-5-nitropyrimidine,
4-methoxy-6-tetramethylenedithiocarbamoyl-5-nitropyrimidine,
3,5-dinitro-2-hexamethylenedithiocarbamoylbenzamide,
3,5-dinitro-2-tetramethylenedithiocarbamoylbenzamide,
3,5-dinitro-2-tetramethylenedithiocarbamoylpyridine,
3,5-dinitro-2-hexamethylenedithiocarbamoylbenzonitril, and
4-methylamino-6-heptamethylenedithiocarbamoyl-5-nitropyrimidine,
   furthermore
3,5-dinitro-2-dimethyldithiocarbamoylbenzonitrile,
3,5-dinitro-2-diethyldithiocarbamoylbenzonitrili,
3,5-dinitro-2-methylethyldithiocarbamoylbenzonitril,
3,5-dinitro-2-methylpropyldithiocarbamoylbenzonitrile,
3,5-dinitro-2-methylisopropyldithiocarbamoylbenzonitril,
3,5-dinitro-2-methylisobutyldithiocarbamoylbenzonitrile,
3,5-dinitro-2-ethylpropyldithiocarbamoylbenzonitril,
3,5-dinitro-2-ethylisopropyldithiocarbamoylbenzonitrile
3,5-dinitro-2-dimethyldithiocarbamoylbenzoamide,
3,5-dinitro-2-diethyldithiocarbamoylbenzoamide,
3,5-dinitro-2-methylethyldithiocarbamoylbenzoamide,
3,5-dinitro-2-methylpropyldithiocarbamoylbenzoamide,
3,5-dinitro-2-methylisopropyldithiocarbamoylbenzoamide,
3,5-dinitro-2-methylisobutyldithiocarbamoylbenzoamide,
3,5-dinitro-2-ethylpropyldithiocarbamoylbenzoamide,
3,5-dinitro-2-ethylisopropyldithiocarbamoylbenzoamide

The substituents mentioned hereinbefore or hereinafter have the following meanings: A saturated or unsaturated, linear or branched aliphatic radical having 1-7 chain members is, for example, a corresponding alkyl, alkenyl or alkinyl radical having 1-6 carbon atoms optionally interrupted by one oxygen or sulphur atom or by NR⁶. A saturated or unsaturated, mono or polyheterocyclic ring with heteroatoms N, S, O is, for example, a five or six membered ring such as morpholino, thiomorpholino, piperazinyl, pyrrolidinyl or piperinyl. Compounds of the formula I wherein R³ and R⁴ together represent a bivalent radical

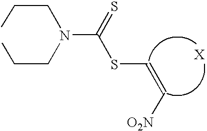

are preferably symmetrical with respect to the symmetric axis dividing the central piperazine ring into two equal halves.

Surprisingly the compounds of the invention exhibit strong antibacterial activities, especially against mycobacteria with minimal inhibitory concentrations (MIC) in the range of 0.1-50 μg/ml for fast growing mycobacteria and of 3.12-12.5 μg/ml for M. tuberculosis, and against multiresistant staphylococci (MRSA) in a MIC range of. 0.4-50 μg/ml.

Thus, the compounds of the invention are useful for the treatment of bacterial infections, especially tuberculosis and other mycobacterial infections, in humans and in animals.

Accordingly, the invention concerns pharmaceutical compositions comprising one or more (ie. at least one) of the compounds of the formula I referred to above.

The invention relates furthermore to a compound of the formula I

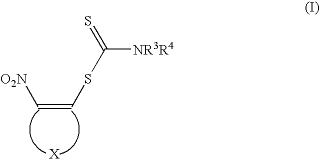

wherein X is a bivalent residue selected from the group consisting of

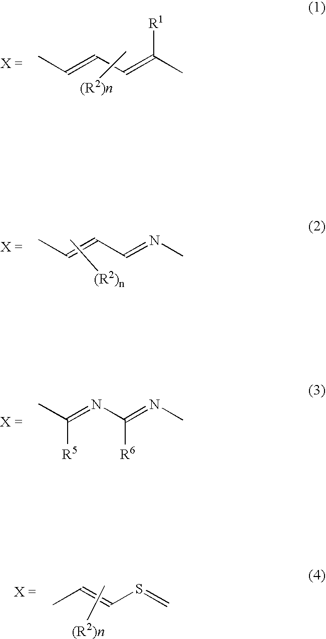

wherein

R¹ is CN, CONR³R⁴, CONHNR³R⁴;

R² is H, NO₂, CN, CONR³R⁴, CONHNR³R⁴, COOR⁵, CHO, halogen or a saturated or unsaturated, linear or branched aliphatic radical having 1-7 chain members optionally substituted by hydroxy; OR⁵, SR⁵, NR³R⁴, SO₂NR³R⁴, trifluoromethyl, phenyl;

n is 0-3;

R³ and R⁴ are, independently from each other, H, a saturated or unsaturated, linear or branched aliphatic radical having 1-7 chain members optionally substituted by hydroxy; cycloalkyl having 3-7 carbon atoms; benzyl, phenyl or naphthyl each unsubstituted or substituted by (R⁵)m wherein m is 1-5; OR⁵; or NR³R⁴ is a saturated or unsaturated, mono or polyheterocyclic ring with heteroatoms N, S, O which is unsubstituted or substituted by (R⁵)m ;

or R³ and R⁴ together represent a bivalent radical

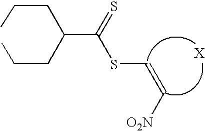

R⁵ is H, or halogen, a saturated or unsaturated, linear or branched aliphatic radical having 1-7 chain members optionally substituted by halogen or hydroxy; benzyl, phenyl, styryl or naphthyl each unsubstituted or substituted by (R⁶)m wherein m is 1-5; COOH, COOR⁵, CONR³N⁴;

R⁶ is H, halogen or a saturated or unsaturated, linear or branched aliphatic radical having 1-7 chain members optionally substituted by halogen or hydroxy; benzyl, phenyl, styryl or naphthyl each unsubstituted or substituted by (R⁶)m; COOH, COOR⁵, CONR³N⁴;

for use in a method for the therapeutic or prophylactic treatment of bacterial infections in mammals, or for the preparation of a pharmaceutical composition for the therapeutic or prophylactic treatment of bacterial infections in mammals, respectively.
Preferred compounds of the formula I for use in such method are those specifically listed above.

The above-referenced compounds are formulated for use by preparing a dilute solution or suspension in pharmaceutically acceptable aqueous, organic or aqueous-organic medium for topical or parenteral administration by intravenous, subcutaneous or intramuscular injection, or for intranasal application; or are prepared in tablet, capsule or aqueous suspension form with conventional excipients for oral administration or as suppositorium.

The compounds can be used in dosages from 0.1-1000 mg/kg body weight.

The examples which follow in the subsequent experimental part serve to illustrate the invention but should not be construed as a limitation thereof.

The structures of the compounds of the invention were established by modes of synthesis and elementary analysis, and by nuclear magnetic resonance and/or mass spectra, X-ray analysis.

### EXPERIMENTAL PART

Starting Materials

Starting chloroaryles are commercial available or were synthesized: 4-chloro-6-R-5-nitropyrimidines were synthesized according to Chesney J. D., Gonzales-Sierra M., Pharm. Res., 1985, p. 145-147 and Clark J., Parvizi B., Colmam R., J. Chem. Soc., Perkin Trans 1, 1976, p. 1004-1007; 3,5-dinitro-2-chlorobenzonitrile and 3,5-dinitro-2-chlorobenzamide were synthesized according to Thiel W., Mayer R., J. Prakt. Chemie, B328, 1986, p. 497-514. Sodium dithiocarbamates were synthesized by classic reaction between carbone disulfide, sodium hydroxide and different amines, and purified by crystallization from ethanol or acetone. Some sodium dithiocarbamates are commercial available.

### Example 1

### 4-isopropylamino-6-diethyldithiocarbamoyl-5-nitropyrimidine (1)

A mixture of 4-isopropylamino-6-chloro-5-nitropyrimidine (0.94 g, 4.3 mmol), sodium diethyldithiocarbamate trihydrate (1.05 g, 4.6 mmol) and ethanol (25 mL) was mixed for 24 hours at room temperature. Reaction mixture was diluted by water (50 mL), yellow solid was filtered and purified by crystallization from ethanol to give 0.8 g of pure title compound as an yellow crystalline solid, m.p. 165-167° C.

Anal. Calcd. for C₁₂H₁₇N₅O₂S₂: C, 44.02; H, 5.23; N, 21.39; S, 19.59

Found: C, 44.31; H, 5.17; N, 21.52; S, 19.78

### Example 2

### 4-cyclopropylamino-6-diethyldithiocarbamoyl-5-nitropyrimidine (2)

Following the procedure of Example 1. Yellow crystalline solid, m.p. 143-145° C.

Anal. Calcd. for C₁₂H₁₇N₅O₂S₂: C, 44.02; H, 5.23; N, 21.39; S, 19.59

Found: C, 44.17; H, 5.31; N, 21.39; S, 19.38

### Example 3

### 3,5-dinitro-2-diethyldithiocarbamoylbenzonitril (3)

A mixture of 3,5-dinitro-2-chlorobenzonitrile (1.5 g, 6.6 mmol), sodium diethyldithiocarbamate trihydrate (1.6 g, 7.1 mmol) and ethanol (40 mL) was mixed for 2 hours at room temperature. Reaction mixture was treated by active charcoal, filtered off. Mother liquid was diluted by water (80 mL), yellow solid was filtered and purified by consecutive crystallization from i-propanol and acetone/water to give 1.2 g of pure title compound as an light yellow crystalline solid, m.p. 151-153° C.

Anal. Calcd. for C₁₂H₁₂N₄O₄S₂: C, 42.34; H, 3.55; N, 16.46; S, 18.84

Found: C, 42.32; H, 3.46; N, 16.41; S, 18.69

### Example 4

### 4-methylamino-6-dimethyldithiocarbamoyl-5-nitropyrimidine (7)

A mixture of 4-methylamino-6-chloro-5-nitropyrimidine (0.7 g, 3.7 mmol), sodium dimethyldithiocarbamate dihydrate (0.7 g, 3.9 mmol), ethanol (15 mL) and acetone (15 mL) was mixed for 4 hours at room temperature. Reaction mixture was diluted by water (60 mL), yellow solid was filtered and purified by crystallization from ethanol to give 0.5 g of pure title compound as an yellow crystalline solid, m.p. 136-137° C.

Anal. Calcd. for C₈H₁₁N₅O₂S₂: C, 35.15; H, 4.06; N, 25.62; S, 23.46

Found: C, 34.95; H, 4.11; N, 25.53; S, 23.58

### Example 5

### 4-methylamino-6-heptamethylenedithiocarbamoyl-5-nitropyrimidine (20)

Following the procedure of Example 4. Yellow crystalline solid, m.p. 180-182° C.

Anal. Calcd. for C₁₁H₁₅N₅O₂S₂: C, 42.16; H, 4.82; N, 22.35; S, 20.46

Found: C, 42.11; H, 5.01; N, 22.42; S, 20.49

### Example 6

### 4-methylamino-6-tetramethylenedithiocarbamoyl-5-nitropyrimidine (12)

Following the procedure of Example 4. Yellow crystalline solid, m.p. 213-215° C.

Anal. Calcd. for C₁₀H₁₃N₅O₂S₂: C, 40.12; H, 4.38; N, 23.39; S, 21.42

Found: C, 40.23; H, 4.37; N, 23.47; S, 21.35

### Example 7

### 4-methylhydroxyamino-6-diethyldithiocarbamoyl-5-nitropyrimidine (5)

Following the procedure of Example 4. Yellow crystalline solid, m.p. 92-94° C.

Anal. Calcd. for C₁₀H₁₅N₅O₃S₂: C, 37.84; H, 4.76; N, 22.07; S, 20.21

Found: C, 37.81; H, 4.57; N, 22.01; S, 20.33

### Example 8

### 4-dimethylamino-6-dimethyldithiocarbamoyl-5-nitropyrimidine (8)

Following the procedure of Example 4. Yellow crystalline solid, m.p. 135-137° C.

Anal. Calcd. for C₉H₁₃N₅O₂S₂: C, 37.62; H, 4.56; N, 24.37; S, 22.32

Found: C, 37.66; H, 4.48; N, 24.49; S, 22.39

### Example 9

### 4-dimethylamino-6-tetramethylenedithiocarbamoyl-5-nitropyrimidine (13)

Following the procedure of Example 4. Yellow crystalline solid, m.p. 130-132° C.

Anal. Calcd. for C₁₁H₁₅N₅N₅O₂S₂: C, 42.16; H, 4.82; N, 22.35; S, 20.46

Found: C, 42.23; H, 4.88; N, 22.36; S, 20.23

### Example 10

### 3,5-dinitro-2-tetramethylendithiocarbanoylthiophene, (28)

A mixture of 3,5-dinitro-2-chlorothiophene (0.5 g, 2.4 mmol), sodium tetramethylendithiocarbamate dihydrate (0.6 g, 2.9 mmol) and ethanol (40 mL) was mixed for 5 hours at room temperature. Reaction mixture was diluted by water (100 mL), light tan yellow solid was filtered and purified by crystallization from mixture EtOH/H2O to give 0.4 g of pure title compound as an yellow crystalline solid, m.p. 178-180° C.

Anal. Calcd. for C₉H₉N₃O₄S₃: C, 33.85; H, 2.84; N, 13.16; S, 30.12

Found: C, 33.93; H, 2.71; N, 13.27; S, 30.16

### Example 11

### 3,5-dinitro-2-dimethyldithiocarbamoylbenzonitrile (9)

A mixture of 3,5-dinitro-2-chlorobenzonitrile (2.0 g, 8.8 mmol), sodium dimethyldithiocarbamate dihydrate (1.7 g, 9.5 mmol) and ethanol (50 mL) was mixed for 0.5 hour at room temperature. Reaction mixture was diluted by water (100 mL), yellow solid was filtered and purified by consecutive crystallization from ethanol with addition of active charcoal to give 1.4 g of pure title compound as an orange solid, m.p. 147-149° C.

Anal. Calcd. for C₁₀H₈N₄O₄S₂: C, 38.46; H, 2.58; N, 17.94; S, 20.53

Found: C, 38.61; H, 2.58; N, 17.93; S, 20.52

### Example 12

### 3,5-dinitro-2-hexamethylenedithiocarbamoylbenzonitrile (19)

Following the procedure of Example 11. Yellow crystalline solid, m.p. 148-150° C.

Anal. Calcd. for C₁₄H₁₄N₄O₄S₂: C, 45.62; H, 3.85; N, 15.29; S, 17.50

Found: C, 45.56; H, 3.87; N, 15.37; S, 17.42

### Example 13

### 3,5-dinitro-2-tetramethylenedithiocarbamoylbenzonitrile, (22)

Following the procedure of Example 11. Yellow crystalline solid, m.p. 131-134° C.

Anal. Calcd. for C₁₂H₁₀N₄O₄S₂: C, 42.60; H, 2.98; N, 16.56; S, 18.95

Found: C, 42.67; H, 3.08; N, 16.63; S, 19.04

### Example 14

### 3,5-dinitro-2-methylethyldithiocarbamoylbenzonitrile, (23)

Following the procedure of Example 11. Orange crystalline solid, m.p. 112-114° C.

Anal. Calcd. for C₁₁H₁₀N₄O₄S₂: C, 42.34; H, 3.55; N, 16.46; S, 18.84

Found: C, 42.49; H, 3.62; N, 16.34; S, 18.73

### Example 15

### 3,5-dinitro-2-methylethyldithiocarbamoylbenzonitrile, (24)

Following the procedure of Example 11. Yellow crystalline solid, m.p. 167-169° C.

Anal. Calcd. for C₁₂H₁₂N₄O₄S₂: C, 40.48; H, 3.09; N, 17.17; S, 19.65

Found: C, 40.51; H, 3.03; N, 17.28; S, 19.53

### Example 16

### 4-methoxy-6-dimethyldithiocarbamoyl-5-nitropyrimidine (14)

A solution of 4-methoxy-6-chloro-5-nitropyrimidine (0.5 g, 2.6 mmol), sodium dimethyldithiocarbamate dihydrate (0.6 g, 3.3 mmol) and ethanol (40 mL) was mixed for 1 hour at room temperature. Reaction mixture was diluted by water (80 mL), light yellow solid was filtered and purified by crystallization from methanol to give 0.4 g of pure title compound as an bright yellow crystalline solid, m.p. 122-125° C.

Anal. Calcd. for C₈H₁₀N₄O₃S₂: C, 35.03; H, 3.67; N, 20.42; S, 23.38

Found: C, 34.93; H, 4.02; N, 20.50; S, 23.42

### Example 17

### 4-methoxy-6-tetramethylenedithiocarbamoyl-5-nitropyrimidine (15)

Following the procedure of Example 16. Yellow crystalline solid, m.p. 147-149° C.

Anal. Calcd. for C₁₀H₁₂N₄O₃S₂: C, 39.99; H, 4.03; N, 18.65; S, 21.35

Found: C, 39.98; H, 4.06; N, 18.73; S, 21.32

### Example 18

### 4-methylamino-6-diethyldithiocarbamoyl-5-nitropyrimidine (4)

Following the procedure of Example 12. Yellow crystalline solid, m.p. 147-150° C.

Anal. Calcd. for C₁₀H₁₅N₅O₂S₂: C, 39.85; H, 5.02; N, 23.24; S, 21.28

Found: C, 39.89; H, 4.97; N, 23.33; S, 21.20

### Example 19

### 4-methoxy-6-methylethyldithiocarbamoyl-5-nitropyrimidine, (25)

Following the procedure of Example 16. Yellow crystalline solid, m.p. 154-156° C.

Anal. Calcd. for C₉H₁₂N₄O₃S₂: C, 37.49; H, 4.19; N, 19.45; S, 22.24

Found: C, 37.38; H, 4.10; N, 19.55; S, 22.37

### Example 20

### 4-ethoxy-2-methyl-6-diethyldithiocarbamoyl-5-nitropyrimidine (6)

Following the procedure of Example 16. Yellow crystalline solid, m.p. 108-110° C.

Anal. Calcd. for C₁₂H₁₈N₄O₃S₂: C, 43.62; H, 5.49; N, 16.96; S, 19.41

Found: C, 43.74; H, 5.45; N, 16.82; S, 20.01

### Example 21

### 4-ethoxy-2-methyl-6-tetramethylenedithiocarbamoyl-5-nitropyrimidine (10)

Following the procedure of Example 16. Yellow crystalline solid, m.p. 100-102° C.

Anal. Calcd. for C₁₀H₁₃N₅O₂S₂: C, 40.12; H, 4.38; N, 23.39; S, 21.42

Found: C, 40.23; H, 4.37; N, 23.47; S, 21.35

### Example 22

### 4-propoxy-6-dimethyldithiocarbamoyl-5-nitropyrimidine (11)

Following the procedure of Example 16. Yellow crystalline solid, m.p. 107-109° C.

Anal. Calcd. for C₁₀H₁₄N₄O₃S₂: C, 39.72; H, 4.67; N, 18.53; S, 21.21

Found: C, 40.04; H, 4.65; N, 18.42; S, 21.56

### Example 23

### 3,5-dinitro-2-hexamethylenedithiocarbamoylbenzamide (16)

A solution of 3,5-dinitro-2-chlorobenzamide (0.7 g, 2.9 mmol), sodium hexamethylenedithiocarbamate dihydrate (0.7 g, 3.0 mmol) and ethanol (50 mL) was mixed for 0.5 hour at room temperature. Reaction mixture was partitioned between water (100 mL) and ethyl acetate (50 mL). The aqueous layer was extracted twice with 30 mL portions of ethyl acetate. The combined organic extracts were washed with water, brine, dried (Na₂SO₄), and concentrated in vacuo to provide brown oil. Its treatment by water provide yellow solid was filtered and purified by consecutive crystallization from ethanol to give 0.6 g of pure title compound as an yellow solid, m.p. 187-189° C.

Anal. Calcd. for C₁₄H₁₆N₄O₅S₂: C, 43.74; H, 4.20; N, 14.57; S, 16.68

Found: C, 43.82; H, 3.99; N, 14.77; S, 16.57

### Example 24

### 3,5-dinitro-2-tetramethylenedithiocarbamoylbenzamide (17)

A solution of 3,5-dinitro-2-chlorobenzamide (1.0 g, 4.0 mmol), sodium tetramethylenedithiocarbamate dihydrate (0.9 g, 4.4 mmol) and acetone (40 mL) was mixed for 0.5 hour at room temperature. Reaction mixture was diluted by water (80 mL), yellow solid was filtered and purified by crystallization from i-propanol to give 0.9 g of pure title compound as an light yellow crystalline solid, m.p. 116-118° C.

Anal. Calcd. for C₁₂H₁₂N₄O₅S₂: C, 40.44; H, 3.39; N, 15.72; S, 18.00

Found: C, 40.57; H, 3.42; N, 15.64; S, 17.87

### Example 25

### 3,5-dinitro-2-(1,4-dioxa-8-azaspiro[4,5]decane)dithiocarbamoylbenzamide, (26)

Following the procedure of Example 23. Yellow crystalline solid, m.p. 144-146° C.

Anal. Calcd. for C₁₀H₁₂N₄O₃S₂: C, 42.05; H, 3.76; N, 13.08; S, 14.97

Found: C, 42.09; H, 3.66; N, 13.13; S, 14.84

### Example 26

### 3,5-dinitro-2(1,4-thiazolan)dithiocarbamoylbenzamide, (27)

Following the procedure of Example 23. Yellow crystalline solid, m.p. 123-126° C.

Anal. Calcd. for C₁₁H₁₀N₄O₅S₃: C, 35.29; H, 2.69; N, 14.96; S, 25.69

Found: C, 35.14; H, 2.61; N, 15.02; S, 25.73

### Example 27

### 3,5-dinitro-2-tetramethylenedithiocarbamoylpyridine (18)

A mixture of 3,5-dinitro-2-chloropyridine (0.5 g, 2.4 mmol), sodium tetramethylenedithiocarbamate dihydrate (0.6 g, 2.9 mmol) and ethanol (40 mL) was mixed for 3 hours at room temperature. Reaction mixture was diluted by water (80 mL), light tan yellow solid was filtered and purified by crystallization from mixture EtOH/DMF to give 0.5 g of pure title compound as an yellow crystalline solid, m.p. 157-159° C.

Anal. Calcd. for C₁₀H₁₀N₄O₄S₂: C, 38.21; H, 3.21; N, 17.82; S, 20.40

Found: C, 38.03; H, 3.22; N, 17.79; S, 20.44

### Example 28

### 3,5-dinitro-2-dimethyldithiocarbamatethiophene, (29)

Following the procedure of Example 27. Light yellow crystalline solid, m.p. 169-171° C.

Anal. Calcd. for C₇H₇N₃O₄S₃: C, 28.66; H, 2.41; N, 14.32; S, 32.79

Found: C, 28.54; H, 2.33; N, 14.29; S, 32.90

### Example 29

### 3-nitro-5-methylaminosulfonyl-2-tetramethylendithiocarbamoylbenzamide, (30)

Following the procedure of Example 27. Yellow crystalline solid, m.p. 136-138° C.

Anal. Calcd. for C₈H₁₁N₃O₄S₄: C, 28.14; H, 3.25; N, 12.31; S, 37.56

Found: C, 28.25; H, 3.37; N, 12.26; S, 37.47

### Example 30

### 2-methyl-4-methylamino-5-nitro-6-methylethyldithiocarbamoylpyrimidine, (21)

Following the procedure of Example 27. Yellow crystalline solid, m.p. 172-174° C.

Anal. Calcd. for C₁₀H₁₅N₅O₂S₂: C, 39.85; H, 5.02; N, 23.24; S, 21.28

Found: C, 40.11; H, 5.12; N, 23.29; S, 21.41

### Example 31

Determination of the inhibitory activity of the compounds of the invention against staphylococci and mycobacteria.

The antibacterial activities of the compounds against multiresistent staphylococci (MRSA) strains 134/93 and 994/93 as well as against Mycobacterium smegmatis SG 987, M. aureum SB66, M. vaccae IMET 1010670 and M. fortuitum B were tested by determination of minimal inhibitory concentrations (MIC) by the micro broth dilution method in Mueller-Hinton broth (Difco) according to the NCCLS guidelines [National Committee for Clinical Laboratory Standards: Methods for dilution antimicrobial susceptibility tests for bacteria that grow aerobically; 4th Ed.; Villanova, Ed.; Approved standard Document M7-A4. NCCLS, (1997)]. The results are presented in Table 1. Activity against M. tuberculosis H37Rv and two drug-resistant clinical isolates was tested by the following method for determination of minimal inhibitory concentrations (MIC) and minimal bactericidal concentrations (MBC):

Strains were inoculated onto solid Lowenstein-Jensen medium. After 21 days, the cultures grown were used to prepare an inoculum suspension corresponding to 5×10⁸ microbial cells/ml). With 0.2 ml of that suspension tubes with 2 ml liquid Shkolnikova medium containing corresponding concentrations of compounds under study—from 100.0 to 0.195 μg/ml. were inoculated. After 14 days of incubation at 37° C. the tubes with liquid medium were centrifuged for 15 min. at 3000 RPM. After discarding the supernatant, the sediment was resuspended in 0.8 ml of sterile 0.9% NaCl. 0.1 ml of the suspension was used to prepare smears subsequently stained by the Ziehl-Neelsen method. The remaining sediment was inoculated in 0.2 ml volumes into three tubes with solid drug free Lowenstein-Jensen medium to determine minimal bactericidal concentrations (MBC). The results were read after 21-28 days of cultivation at 37° C. Controls were tubes cultured with test-strains not treated with the studied agents.

Minimal bactericidal concentration of drugs (MBC) was considered as the drug concentration completely inhibiting the growth of mycobacteria on the solid medium. The bacteriostatic effect (MIC) was characterized by the presence of only individual mycobacteria in the smear and a strong decrease in the number of colonies grown on solid media compared to the controls. The results are presented in Table 2 as mean values of the three strains.

TABLE 1
Antimicrobial activity of compounds as of the formula I determined
by minimal inhibitory concentrations MIC [μg/ml]

| Subst. | | | | | | M. |
|---|---|---|---|---|---|---|
| Nr. | 134/93 | 994/93 | SG 987 | SB 66 | 10670 | fort. |
| 1 | 3.12 | 25 | 12.5 | 25 | 3.12 | 25 |
| 2 | 1.56 | 3.12 | 6.25 | 12.5 | 3.12 | 6.25 |
| 3 | 3.12 | 6.25 | 12.5 | 12.5 | 3.12 | 6.25 |
| 4 | 0.78 | 1.56 | 6.25 | 6.24 | 1.56 | 6.25 |
| 5 | 1.56 | 3.12 | 6.25 | 12.5 | 3.12 | 6.25 |
| 6 | 3.12 | 3.12 | 12.5 | 12.5 | 3.12 | 12.5 |
| 7 | 1.56 | 3.12 | 12.5 | 6.25 | 3.12 | 6.25 |
| 8 | 3.12 | 6.52 | 12.5 | 6.25 | 3.12 | 12.5 |
| 9 | 0.4 | 0.4 | 3.12 | 1.56 | 3.12 | 6.25 |
| 10 | 3.12 | 0.8 | 12.5 | 25 | 12.5 | 50 |
| 11 | 0.8 | 0.8 | 6.25 | 1.56 | 0.8 | 1.56 |
| 12 | n.d. | n.d. | 3.12 | 1.56 | 0.8 | 0.8 |
| 13 | 3.12 | 1.56 | 3.12 | 3.12 | 1.56 | 1.56 |
| 14 | 6.25 | 3.12 | 3.12 | 0.8 | 3.12 | 0.8 |
| 15 | 3.12 | 3.12 | 3.12 | 1.56 | 0.8 | 1.56 |
| 16 | 6.25 | 12.5 | 6.25 | 6.25 | 0.1 | 0.8 |
| 17 | 25 | 12.5 | 50 | 6.25 | 0.4 | 6.25 |
| 18 | 1.56 | 12.5 | 6.25 | 3.12 | 3.12 | 3.12 |

n.d.: not determined

TABLE 2
Antimicrobial activity of compounds of the formula I against
Mycobacterium tuberculosis as determined by minimal inhibitory
concentrations (MIC) and minimal bactericidal concentrations (MBC)

| | MIC | MBC |
|---|---|---|
| Subst. Nr. | (μg/ml) | (μg/ml) |
| 1 | 31.3 | 100.0 |
| 2 | 20.8 | 29.1 |
| 3 | 5.37 | 7.29 |
| 4 | 6.25 | 12.5 |
| 5 | 9.38 | 12.5 |
| 6 | 20.8 | 41.7 |
| 7 | 7.78 | 10.42 |
| 8 | 7.81 | 12.5 |
| 9 | 4.78 | 6.25 |
| 10 | 5.21 | 8.33 |
| 11 | 9.37 | 12.5 |
| 12 | 3.64 | 8.33 |
| 13 | 3.12 | 6.25 |

1. Use of compounds of the general formula I 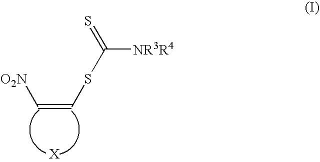
wherein X is a bivalent residue selected from the group consisting of 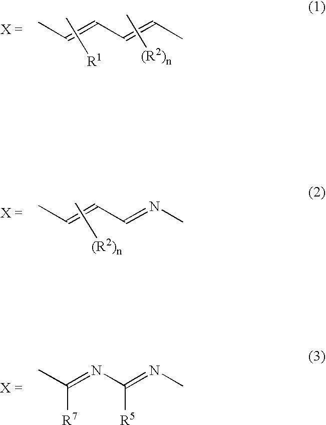
wherein
R′CN, CONR³R⁴, CONHNR³R⁴;
R² is H, NO₂, CN, CONR³R⁴, CONHNR³R⁴, COOR⁵, CHO, halogen or a saturated or unsaturated, linear or branched aliphatic radical having 1-7 chain members, a saturated or unsaturated, linear or branched alkanol radical having 1-8 chain member, OR⁵, SR⁵, NR³R⁴, SO₂NR³R⁴, trifluoromethyl, phenyl;
n is 0-3;
R³ and R⁴ are, independently from each other, H, a saturated or unsaturated, linear or branched aliphatic radical having 1-7 chain members, cycloalkyl having 3-7 carbon atoms; a saturated or unsaturated, linear or branched alkanol radical having 1-8 chain member; benzyl, phenyl or naphthyl substituted by (R⁵)m wherein m is 0-7; OR⁵; or NR³R⁴ is morpholino, a saturated or unsaturated, mono or polyheterocyclic residue with heteroatoms N, S, O and substituted by (R⁵)m; or R³ and R⁴ together represent a bivalent radical —(CH₂)ₘ— wherein m is 2-7, or a bivalent radical —(CH₂CH₂)NR⁶(CH₂CH₂)—; or R³ and R⁴ together represent a bivalent radical 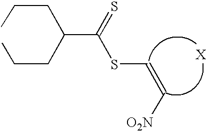
R⁵ is H, or a saturated or unsaturated, linear or branched aliphatic radical having 1-7 chain members, trifluoromethyl, benzyl or phenyl; or halogen, a saturated or unsaturated, halogenated or unhalogenated, linear or branched aliphatic radical having 1-7 chain members, a saturated or unsaturated, linear or branched alkanol radical having 1-8 chain members, benzyl, phenyl, stiryl or naphtyl, each unsubstituted or substituted by (R⁶)m; COOH, COOR⁵, CONR N;
R⁶ is H, halogen or a saturated or unsaturated, linear or branched aliphatic radical having 1-7 chain members, a saturated or unsaturated, halogenated or unhalogenated linear or branched alkanol radical having 1-8 chain member, trifluoromethyl, benzyl, phenyl, stiryl or naphtyl, each unsubstituted or substituted by (R⁶)m; COOH, COOR⁵, CONR³N⁴;
R⁷ is H, halogen or a saturated or unsaturated, linear or branched aliphatic radical having 1-7 chain members, OR⁵, SR⁵, NR³R⁴, trifluoromethyl, phenyl, stiryl, each unsubstituted or substituted by (R⁶)m;
for the manufacture of pharmaceutical preparations for the therapeutic or prophylactic treatment of bacterial infections.

2. A compound of the formula I 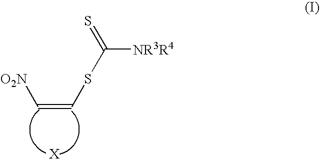
wherein X is a bivalent residue selected from the group consisting of 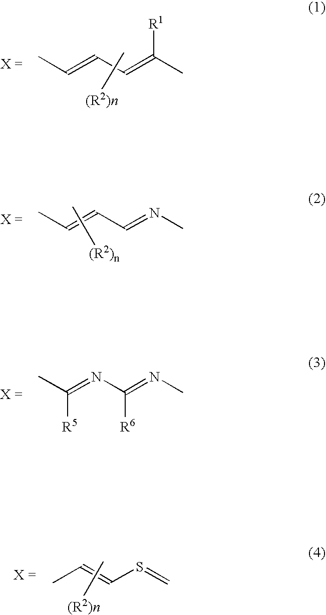
wherein
R¹ is CN, CONR³R⁴, CONHNR³R⁴;
R² is H, NO₂, CN, CONR³R⁴, CONHNR³R⁴, COOR⁵, CHO, halogen or a saturated or unsaturated, linear or branched aliphatic radical having 1-7 chain members optionally substituted by hydroxy; OR⁵, SR⁵, NR³R⁴, SO₂NR³R⁴, trifluoromethyl, phenyl;
n is 0-3;
R³ and R⁴ for X=1 are, independently from each other, H, an unsaturated, linear or branched aliphatic radical having 1-7 chain members; cycloalkyl having 3-7 carbon atoms; a saturated or unsaturated, linear or branched alkyl radical having 1-8 chain member substituted by hydroxy; benzyl, phenyl or naphthyl each unsubstituted or substituted by (R⁵)m wherein m is 1-5; OR⁵; or NR³R⁴ is a saturated or unsaturated, mono or polyheterocyclic ring with heteroatoms N, S, O which is unsubstituted or substituted by (R⁵)m ;
R³ and R⁴ for X=2-4 are, independently from each other, H, a saturated or unsaturated, linear or branched aliphatic radical having 1-7 chain members optionally substituted by hydroxy; cycloalkyl having 3-7 carbon atoms; benzyl, phenyl or naphthyl each unsubstituted or substituted by (R⁵)m wherein m is 1-5; OR⁵; or NR³R⁴ is a saturated or unsaturated, mono or polyheterocycles with heteroatoms N, S, O and substituted by (R⁵)m ;
or R³ and R⁴ for X=1-4 together represent a bivalent radical; 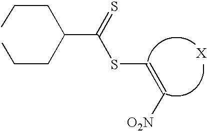
R⁵ is H, or halogen, a saturated or unsaturated, linear or branched aliphatic radical having 1-7 chain members optionally substituted by halogen or hydroxy; benzyl, phenyl, styryl or naphthyl each unsubstituted or substituted by (R⁶)m wherein m is 1-5; COOH, COOR⁵, CONR³N⁴;
R⁶ is H, halogen or a saturated or unsaturated, linear or branched aliphatic radical having 1-7 chain members optionally substituted by halogen or hydroxy; benzyl, phenyl, styryl or naphthyl each unsubstituted or substituted by (R⁶)m; COOH, COOR⁵, CONR³N⁴;
with the exclusion of
3,5-dinitro-2-dimethyldithio-carbamoyl-pyridine, 3,5-dinitro-6-dimethyl-amino-2-dimethyl-dithiocarbamoyl-pyridine, 4-methoxy-6-diethyldithiocarbamoyl-5-nitro-pyrimidine, 4-methoxy-6-dipropyldithiocarbamoyl-5-nitro-pyrimidine, 4-dimethyl-amino-6-diethyldithiocarbamoyl-5-nitropyrimidine, 4-dimethylamino-6-dipropyldithiocarbamoyl-5-nitro-pyrimidine, and 4-methyl-amino-6-diethyldithiocarbamoyl-5-nitro-pyrimidine.

3. A 3,5-dinitro-2-R³R⁴-dithiocarbamoyl-benzonitrile of the formula (I) according to claim 2 wherein X is the residue of the formula (1), R¹ represents CN, R² is NO₂.

4. A 3,5-dinitro-2-R³R⁴-dithiocarbarnoyl-benzamide of the formula (I) according to claim 2 wherein X is the residue of the formula (1), R₁ represents CONR³R⁴, R² is NO₂.

5. A 3,5-dinitro-2-R³R⁴-dithiocarbamoyl-pyridine of the formula (I) according to claim 1 wherein X is the residue of the formula (2), R² is NO₂, n is 1 excluding 3,5-dinitro-2-dimethyl-dithiocarbamoyl-pyridine.

6. A 4-R⁷-2-R⁵-6-R³R⁴-dithiocarbamoyl-5-nitro-pyrimidine of the formula (I) according to claim 2 wherein X is the residue of the formula (3), excluding, 4-methoxy-6-diethyldithio-carbamoyl-5-nitro-pyrimidine, 4-methoxy-6-dipropyldithiocarbamoyl-5-nitro-pyrimidine, 4-dimethylamino-6-diethyldithiocarbamoyl-5-nitro-pyrimidine, 4-dimethylamino-6-dipropyl-dithio-carba-moyl-5-nitro-pyrimidine and 4-methylamino-6-diethyldithiocarbamoyl-5-nitro-pyrimi-dine.

7. A compound of the formula I according to claim 2 selected from the group consisting of
3,5-dinitro-2-dimethyldithiocarbamoylbenzonitrile,
3,5-dinitro-2-diethyldithiocarbamoylbenzonitril,
3,5-dinitro-2-methylethyldithiocarbamoylbenzonitril,
3,5-dinitro-2-methylpropyldithiocarbamoylbenzonitrile,
3,5-dinitro-2-methylisopropyldithiocarbamoylbenzonitril,
3,5-dinitro-2-methylisobutyldithiocarbamoylbenzonitrile,
3,5-dinitro-2-ethylpropyldithiocarbamoylbenzonitril,
3,5-dinitro-2-ethylisopropyldithiocarbamoylbenzonitrile.

8. A compound of the formula I according to claim 2 selected from the group consisting of
3,5-dinitro-2-dimethyldithiocarbamoylbenzoamide,
3,5-dinitro-2-diethyldithiocarbamoylbenzoamide,
3,5-dinitro-2-methylethyldithiocarbamoylbenzoamide,
3,5-dinitro-2-methylpropyldithiocarbamoylbenzoamide,
3,5-dinitro-2-methylisopropyldithiocarbamoylbenzoamide,
3,5-dinitro-2-methylisobutyldithiocarbamoylbenzoamide,
3,5-dinitro-2-ethylpropyldithiocarbamoylbenzoamide,
3,5-dinitro-2-ethylisopropyldithiocarbamoylbenzoamide.

9. A compound of the formula I according to claim 2 in which R³ and R⁴ together represent a bivalent radical of 2,5-dihydropyrrole, 1,2,3,6-tetrahydropyridine, 1,3-thiazolane, 1,4-dioxa-8-azaspiro[4.5]decane.

10. A compound of the formula I according to claim 2 selected from the group consisting of
4-cyclopropylamino-6-diethyldithiocarbamoyl-5-nitropyrimidine,
4-methylhydroxyamino-6-diethyldithiocarbamoyl-5-nitropyrimidine,
4-ethoxy-2-methyl-6-diethyldithiocarbamoyl-5-nitropyrimidine,
4-methylamino-6-dimethyldithiocarbamoyl-5-nitropyrimidine,
4-dimethylamino-6-dimethyldithiocarbamoyl-5-nitropyrimidine,
4-ethoxy-2-methyl-6-tetramethylenedithiocarbamoyl-5-nitropyrimidine,
4-propoxy-6-dimethyldithiocarbamoyl-5-nitropyrimidine,
4-methylamino-6-tetramethylenedithiocarbamoyl-5-nitropyrimidine,
4-dimethylamino-6-tetramethylenedithiocarbamoyl-5-nitropyrimidine,
4-methoxy-6-dimethyldithiocarbamoyl-5-nitropyrimidine,
4-methoxy-6-tetramethylenedithiocarbamoyl-5-nitropyrimidine,
3,5-dinitro-2-hexamethylenedithiocarbamoylbenzamide,
3,5-dinitro-2-tetramethylenedithiocarbamoylbenzamide,
3,5-dinitro-2-tetramethylenedithiocarbamoylpyridine,
3,5-dinitro-2-hexamethylenedithiocarbamoylbenzonitril, and
4-methylamino-6-heptamethylenedithiocarbamoyl-5-nitropyrimidine.

11. A pharmaceutical composition comprising a compound of the formula I according to claim 2.

12. A compound of the formula I 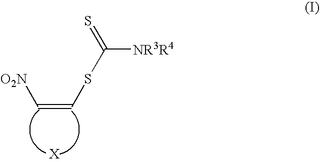
wherein X is a bivalent residue selected from the group consisting of 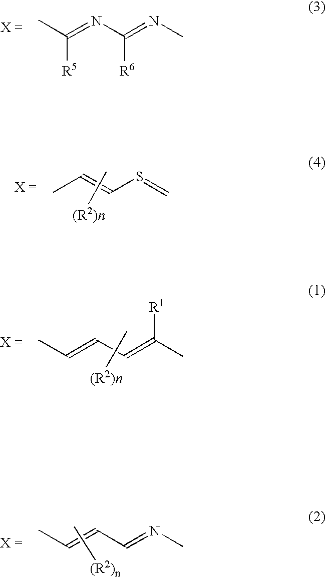
wherein
R¹ is CN, CONR³R⁴, CONHNR³R⁴; R² is H, NO₂, CN, CONR³R⁴, CONHNR³R⁴, COOR⁵, CHO, halogen or a saturated or unsaturated, linear or branched aliphatic radical having 1-7 chain members optionally substituted by hydroxy; OR⁵, SR⁵, NR³R⁴, SO₂NR³R⁴, trifluoromethyl, phenyl;
n is 0-3;
R³ and R⁴ are, independently from each other, H, a saturated or unsaturated, linear or branched aliphatic radical having 1-7 chain members optionally substituted by hydroxy; cycloalkyl having 3-7 carbon atoms; benzyl, phenyl or naphthyl each unsubstituted or substituted by (R⁵)m wherein m is 1-5; OR⁵; or NR³R⁴ is a saturated or unsaturated, mono or polyheterocyclic ring with heteroatoms N, S, O which is unsubstituted or substituted by (R⁵)m ;
or R³ and R⁴ together represent a bivalent radical 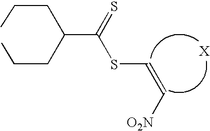
R⁵ is H, or halogen, a saturated or unsaturated, linear or branched aliphatic radical having 1-7 chain members optionally substituted by halogen or hydroxy; benzyl, phenyl, styiyl or naphthyl each unsubstituted or substituted by (R⁶)m wherein m is 1-5; COOH, COOR⁵, CONR³N⁴;
R⁶ is H, halogen or a saturated or unsaturated, linear or branched aliphatic radical having 1-7 chain members optionally substituted by halogen or hydroxy; benzyl, phenyl, styryl or naphthyl each unsubstituted or substituted by (R⁶)m; COOH, COOR⁵, CONR³N⁴;
for use in a method for the therapeutic or prophylactic treatment of bacterial infections in mammals.

13. A pharmaceutical composition comprising a compound of the formula I 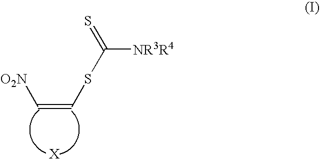
wherein X is a bivalent residue selected from the group consisting of 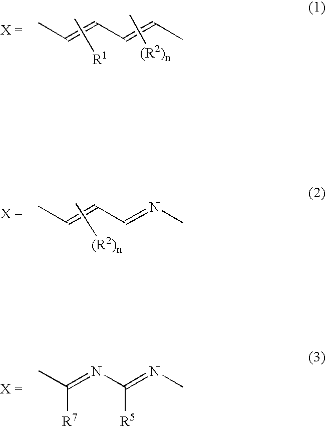
wherein
R¹ is CN, CONR³R⁴, CONHNR³R⁴;
R² is H, NO₂, CN, CONR³R⁴, CONHNR³R⁴₂, COOR⁵, CHO, halogen or a saturated or unsaturated, linear or branched aliphatic radical having 1-7 chain members, a saturated or unsaturated, linear or branched alkanol radical having 1-8 chain member, OR⁵, SR⁵, NR³R⁴, SO₂NR³R⁴, trifluoromethyl, phenyl;
n is 0-3;
R³ and R⁴ are, independently from each other, H, a saturated or unsaturated, linear or branched aliphatic radical having 1-7 chain members, cycloalkyl having 3-7 carbon atoms; a saturated or unsaturated, linear or branched alkanol radical having 1-8 chain member; benzyl, phenyl or naphthyl substituted by (R⁵)m wherein m is 0-7; OR⁵; or NR³R⁴ is morpholino, a saturated or unsaturated, mono or polyheterocyclic residue with heteroatoms N, S, O and substituted by (R⁵)m ; or R³ and R⁴ together represent a bivalent radical —(CH₂)ₘ— wherein m is 2-7, or a bivalent radical —(CH₂CH₂)NR⁶(CH₂CH₂)—; or R³ and R⁴ together represent a bivalent radical 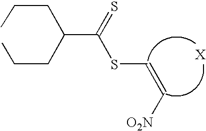
R⁵ is H, or a saturated or unsaturated, linear or branched aliphatic radical having 1-7 chain members, trifluoromethyl, benzyl or phenyl; or halogen, a saturated or unsaturated, halogenated or unhalogenated, linear or branched aliphatic radical having 1-7 chain members, a saturated or unsaturated, linear or branched alkanol radical having 1-8 chain members, benzyl, phenyl, stiryl or naphtyl, each unsubstituted or substituted by (R⁶)m; COOH, COOR⁵, CONR³N⁴;
R⁶ is H, halogen or a saturated or unsaturated, linear or branched aliphatic radical having 1-7 chain members, a saturated or unsaturated, halogenated or unhalogenated linear or branched alkanol radical having 1-8 chain member, trifluoromethyl, benzyl, phenyl, stiryl or naphtyl, each unsubstituted or substituted by (R⁶)m; COOH, COOR⁵, CONR³N⁴;
R⁷ is H, halogen or a saturated or unsaturated, linear or branched aliphatic radical having 1-7 chain members, OR⁵, SR⁵, NR³R⁴, trifluoromethyl, phenyl, stiryl, each unsubstituted or substituted by (R⁶)m.

14. Use according to claim 13, wherein the compound according to formula I is 1,5-dinitro-3-cyano-6-dimethyldithiocarbamoyl-benzene, 3,5-dinitro-2-dimethyldithio-carbamoyl-pyridine, 3,5-dinitro-6-dimethyl-amino-2-dimethyl-dithiocarbamoyl-pyridine, 4-methoxy-6-diethyldithiocarbamoyl-5-nitro-pyrimidine, 4-methoxy-6-dipropyldithiocar-bamoyl-5-nitro-pyrimidine, 4-dimethyl-amino-6-diethyldithiocarbamoyl-5-nitropyrimi-dine, 4-dimethylamino-6-dipropyldithio-carbamoyl-5-nitro-pyrimidine, and/or 4-methyl-amino-6-diethyldithiocarbamoyl-5-nitro-pyrimidine.
